(19) United States
(12) Patent Application Publication
(10) Pub. No.: US 2026/0053366 A1
(43) Pub. Date: Feb. 26, 2026

(54) IMAGING DEVICE BASED ON MULTI-PHOTON DEPTH IMAGING AND IMAGING PROBE THEREOF

(71) Applicants: Beijing Transcend Vivoscope Bio-Technology Co., Ltd., Beijing (CN); Peking University, Beijing (CN)

(72) Inventors: Runlong WU, Beijing (CN); Chunzhu ZHAO, Beijing (CN); Yanhui HU, Beijing (CN); Aimin WANG, Beijing (CN); Heping CHENG, Beijing (CN)

(73) Assignees: Beijing Transcend Vivoscope Bio-Technology Co., Ltd., Beijing (CN); Peking University, Beijing (CN)

(21) Appl. No.: 19/067,541

(22) Filed: Feb. 28, 2025

Related U.S. Application Data
(63) Continuation of application No. PCT/CN2024/120620, filed on Sep. 24, 2024.

(30) Foreign Application Priority Data
Aug. 21, 2024 (CN) 202411155541.1

(51) Int. Cl. A61B 5/00

(52) U.S. Cl. CPC A61B 5/0071; A61B 2503/40

(57) ABSTRACT

Disclosed are an imaging device based on multi-photon depth imaging and an imaging probe thereof, and a miniaturized imaging probe is implemented by using a separated structure. During imaging, the imaging probe may be fixedly connected to an imaging object, and an optical signal inside the imaging object may be collected by exciting a multi-photon effect inside the imaging object by excitation light to detect an internal status in the imaging object.

### CROSS-REFERENCE TO RELATED APPLICATIONS

The present application is a continuation of International Application No. PCT/CN2024/120620, filed on Sep. 24, 2024, which claims priority to Chinese Patent Application No. 202411155541.1, filed on Aug. 21, 2024. The disclosures of the aforementioned applications are hereby incorporated by reference in their entireties.

### TECHNICAL FIELD

The present application relates to the field of multi-photon imaging technologies, and in particular, to an imaging device based on multi-photon depth imaging and an imaging probe thereof.

### BACKGROUND

Multi-photon depth imaging is a technology for depth imaging based on nonlinear optical effects multi-photon. Specifically, multi-photon imaging generates corresponding optical signals by using various nonlinear phenomena and effects generated when a plurality of photons interact simultaneously with a substance to perform depth scanning imaging. For example, the nonlinear optical effects of the multi-photon depth imaging include second harmonic generation (SHG), third harmonic generation (THG), two-photon excitation fluorescence (TPEF), three-photon excitation fluorescence (3PEF), coherent anti-Stokes Raman scattering (CARS), and the like.

In practical applications, because an optical path inside an imaging device of multi-photon imaging is complex, it is often implemented depended on a large-scale device.

### SUMMARY

Therefore, how to implement a multi-photon imaging technology based on a micro device and present a-scale imaging effect is an urgent technical problem to be solved urgently by a person skilled in the art.

In view of this, an embodiment of the present application provides an imaging device that implements miniaturization of a probe through separated design of a probe and a main unit. In addition, an input optical fiber used for transmitting excitation light has a target bandwidth that meets a multi-photon imaging requirement, and is capable of carrying a combined laser beam that includes different wavelength components, so that an imaging effect of the probe is improved and the foregoing technical problem is solved.

According to a first aspect, the present application provides an imaging device based on multi-photon depth imaging, and the imaging device includes at least two laser devices, a coupling module, an input optical fiber, an imaging probe, a beam splitting module, at least two optical detectors and an imaging main unit.

The imaging main unit at least accommodates the at least two laser devices and the coupling module, the laser devices are configured to provide laser beams meeting an imaging requirement, the coupling module is configured to couple laser beams released by the at least two laser devices into a combined laser beam, where laser beams released by laser devices in the at least two laser devices have different wavelengths.

An end of the input optical fiber is connected to the coupling module and the other end is connected to the imaging probe for transmitting the combined laser beam to the imaging probe, where an energy transmission loss of the input optical fiber in a target bandwidth meets an excitation requirement of a multi-photon effect, and a wavelength of each laser beam in the combined laser beam is within the target bandwidth.

The imaging probe is internally provided with an imaging apparatus that is based on multi-photon depth imaging and configured to: convert the combined laser beam into excitation light; focus the excitation light at an internal focusing position of an imaging object to trigger a multi-photon effect at the internal focusing position; collect optical signals generated by the multi-photon effect; and change the internal focusing position to determine optical signals of various positions in a focal plane, where the optical signals of various positions in the focal plane are used for generating an optical image of the imaging object at the focal plane.

The beam splitting module is configured to split the optical signals into at least two optical signal splitting beams, and the optical detector is configured to receive a corresponding optical signal beam and detect a signal intensity of the corresponding optical signal splitting beam, where the optical signal splitting beams reflect signal components of the optical signals at various wavelengths generated by the multi-photon effect, the optical signal splitting beams are in one-to-one correspondence with the optical detectors, and a signal intensity of the optical signal reflects a pixel value of a corresponding pixel in the optical image.

The present application provides a micro multi-photon imaging device, and a miniaturized imaging probe is implemented by using a separated structure. During imaging, the imaging probe may be fixedly connected to an imaging object, and an optical signal inside the imaging object may be collected by exciting a multi-photon effect inside the imaging object by excitation lights to detect an internal status in the imaging object. In particular, an input optical fiber connected to the imaging probe and an imaging main unit in the imaging device provided by the present application has a target bandwidth that meets an excitation requirement of a multi-photon imaging, so that a combined laser beam in the target bandwidth is capable of being carried to simultaneously excite multi-photon effects at different wavelengths to observe different internal structures. In addition, for the imaging device, a coupling module of laser beams are disposed outside the imaging probe, so that the combined laser beam is directly transmitted through the input optical fiber, and thus integration of the imaging probe is improved.

### BRIEF DESCRIPTION OF THE DRAWINGS

To describe the technical solutions in embodiments of the present application more clearly, the following briefly describes the accompanying drawings required in the embodiments. It should be understood that the accompanying drawings in the following description show only some embodiments of the present application, and therefore should not be taken as limiting the scope. A person of ordinary skill in the art may still derive other drawings from these accompanying drawings without creative efforts.

FIG. 1 is a diagram of an application scenario of an imaging device according to some embodiments of the present application.

FIG. 2 is a schematic diagram of photoelectric signal transmission in an imaging device according to some embodiments of the present application.

FIG. 3A and FIG. 3B are schematic diagrams of a structure and performance of input optical fibers provided in some embodiments of the present application.

FIG. 4 is a system block diagram of an imaging device according to some embodiments of the present application.

FIG. 5 is another system block diagram of an imaging device according to some embodiments of the present application.

FIG. 6 is a schematic structural diagram of an imaging probe according to some embodiments of the present application.

FIG. 7 is another schematic structural diagram of an imaging probe according to some embodiments of the present application.

FIG. 8 is a schematic diagram of optical paths of parallel excitation light in an imaging probe according to some embodiments of the present application.

FIG. 9A to FIG. 9C are schematic structural diagrams in which a scanning galvanometer and excitation light are at an acute angle according to some embodiments of the present application.

FIG. 10 is a schematic structural diagram of a zoom imaging probe according to some embodiments of the present application.

FIG. 11A to FIG. 11C are schematic structural diagrams of imaging probes with optogenetic stimulation according to some embodiments of the present application.

### DETAILED DESCRIPTION OF THE EMBODIMENTS

As mentioned above, in multi-photon imaging, it is often depended on multi-photon linearity triggered by excitation light to obtain a corresponding optical signal. For example, sub-harmonic generation (SHG) is a second-order multi-photon phenomenon in which two photons with a same frequency are combined after interacting with a non-linear material, to form a new photon with twice the frequency. For another example, a three-photon excitation fluorescence (3PEF) requires three photons to be simultaneously excited to produce a fluorescence signal.

In an actual application, based on a characteristic of multi-photon imaging, the multi-photon imaging is often applied to depth imaging, that is, excitation light which includes a plurality of photons generally needs to be focused at a specific depth, to trigger a corresponding multi-photon effect, to generate a corresponding optical signal. Therefore, there are certain requirements on a frequency and a power of the excitation light in multi-photon. In addition, further considering that multi-photon is often dependent on excitation light of a specific frequency band, the multi-photon imaging is often implemented only based on excitation light of a single frequency band in practical applications. Especially in an imaging process of a miniaturized device, excitation light propagation cannot be propagated in consideration of complexity of an internal space of the imaging device.

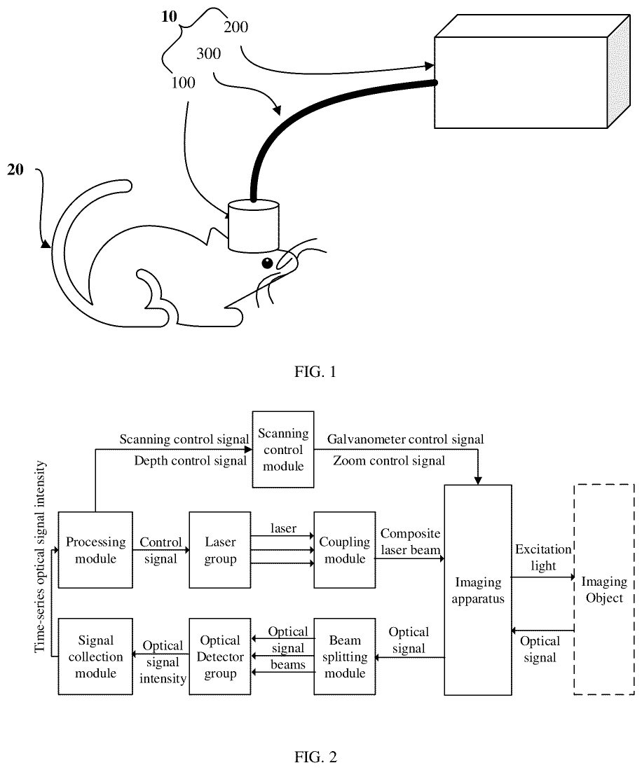

To further describe a practical application case of a miniaturized device in a multi-photon imaging technology, the present application provides a schematic diagram of an application scenario of an imaging device (FIG. 1). The application scenario shown in FIG. 1 may reflect a process in which an imaging device performs optical brain imaging on a mouse.

As shown in FIG. 1, in the application scenario of the imaging device, an imaging device 10 and an imaging object 20 may be included.

The imaging device 10 is an execution body of multi-photon imaging (for example, the imaging device 10 may be configured as a multi-photon microscope), and may release excitation light to trigger a multi-photon effect (such as a multi-photon fluorescence effect) inside the imaging object 20, so that an optical signal of the imaging object 20 excited by the excitation light is acquired to generate a fluorescence image of the imaging object 20.

Illustratively, the imaging device 10 may focus, based on a multi-photon imaging principle, a plurality of photons of the excitation light on a point at a target focusing depth region inside the imaging object 20, to acquire a fluorescence signal of this point. Then a scanning technology is used to perform plane scanning by changing a focusing position at the target focusing depth region, to acquire fluorescence signals of various positions at the target focusing depth region inside the imaging object 20 to determine the fluorescence image.

The imaging object 20 may refer to an object for multi-photon imaging as required. For example, the imaging object 20 may be an ex vivo sample, a living body sample, or the like. Illustratively, the imaging object 20 in the present application may be a mouse, a rabbit, a bird, a non-human primate macaque, a marmoset, or another imaging object. For the imaging object 20, imaging may be generally performed on an internal structure (a brain nerve, a spine nerve, or the like) of the imaging object 20.

As shown in FIG. 1, the imaging object 20 in FIG. 1 may be a mouse, and specifically multi-photon imaging may be performed on a brain of the mouse. The imaging device 10 may be fixedly connected to a brain of the imaging object 20. The imaging object 20 may be free to move, and the imaging device 10 may periodically scan the imaging object 20 during free movement of the imaging object 20, to acquire an optical signal of each position in a specific focal plane of the brain of the imaging object 20 to determine a time-series optical image of the position. The collected optical image may generally include a neural synapse of the position.

It should be noted that before the imaging object 20 is fixedly connected to the imaging device 10, adaptive processing may be performed on the imaging object 20. For example, skin preparation may be performed on a fixed connection position on the imaging object 20 at where the imaging object 20 is connected to the imaging device 10. For another example, for the mouse shown in FIG. 1, a cranial window may be set in the brain of the mouse before the fixed connection, to improve an imaging effect. For another example, a fluorescence probe may be injected into the imaging object 20 in advance for performing multi-photon fluorescence imaging based on the fluorescence probe.

To miniaturize a front-facing imaging device, the imaging device 10 provided in this specification may be disposed in a separated manner, so that an imaging probe 100 and an imaging main unit 200 may be further included. The imaging probe 100 and the imaging main unit 200 may be communicatively connected based on a photoelectric composite cable 300.

The imaging probe 100 may be an apparatus for releasing excitation light and detecting an optical signal. The imaging probe 100 may convert, through an optical component, a laser beam generated by a laser source into excitation light which is focused on a focal plane and has a scanning capability. In some embodiments, the imaging probe 100 may be a contact imaging probe.

The imaging main unit 200 may be a device set that cannot be integrated into the imaging probe 100, and may be specifically configured to provide a laser beam and perform imaging based on an optical signal. The imaging main unit 200 may generally include a laser device, a fluorescence collection apparatus, and a scan collection controller.

The photoelectric composite cable 300 may be a communication cable between the imaging probe 100 and the imaging main unit 200, and is configured to implement data transmission of an optical signal and an electrical signal between the imaging main unit 200 and the imaging probe 100. The photoelectric composite cable 300 may transmit excitation light generated by the imaging main unit 200 to the imaging probe 100, and transmit an optical signal collected by the imaging probe 100 to the imaging main unit 200 to form an optical image.

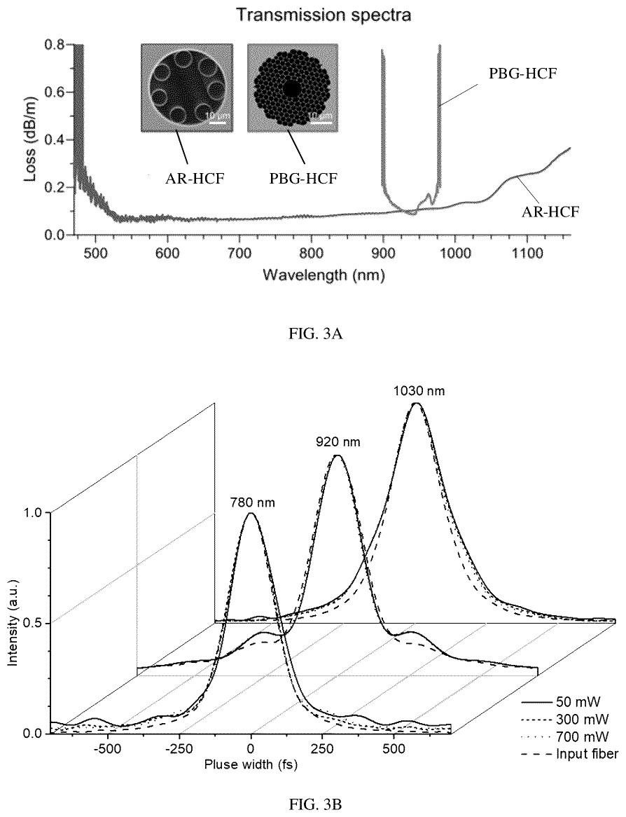

Multi-photon imaging (for example, multi-photon fluorescence imaging) for the interior of the imaging object 20 may be implemented based on the imaging device 10. However, considering that currently spectrum bandwidth of optical fibers is limited and the imaging device 10 always requires a highly stable laser beam with a relatively high power, an optical fiber usually can be used for transmitting only a laser beam with a single wavelength. For details, refer to FIG. 3. Consequently, an optical fiber that transmits excitation light in the imaging device 10 is generally used for transmitting only excitation light with a specific wavelength, and the imaging device 10 often performs multi-photon imaging based on only excitation light of a specific wavelength.

However, in practical applications, there is a need for simultaneous imaging of a plurality of substances. For example, when multi-photon imaging is performed on a brain of a mouse, calcium ions, plaques, and neural synapses in the brain are all objects required to be observed, and often require excitation light of different wavelengths.

To implement excitation light of a plurality of wavelengths, in a related technology, multi-photon imaging of excitation light of a plurality of wavelengths may be implemented by switching laser sources. That is, when multi-photon scanning is performed, laser sources connected to an optical fiber may be switched at a main unit, so that different wavelengths are provided at different times to implement multi-photon imaging of a plurality of wavelengths.

However, when time division multiplexing is used to perform imaging, optical signals corresponding to different wavelengths are not synchronized in time. When a specific parameter (such as a transient change of calcium) is analyzed, this method cannot meet an actual requirement. In addition, considering a bandwidth of an optical fiber, when the foregoing method is performed, an excitation efficiency of photons may be affected, and thus some fluorescent substances are not excited under an optimal excitation condition, which affects imaging quality.

Moreover, in conventional fluorescence imaging, there is also a technology of simultaneously transmitting different fluorescent light sources in one cable. Specifically, different optical signals may be transmitted by using a composite optical fiber to form a composite optical field. However, a conventional fluorescence imaging principle is completely different from a multi-photon depth imaging principle, and transmission of the foregoing composite optical fiber can only provide a field light source required for conventional fluorescence imaging, and cannot meet a requirement of multi-photon imaging.

Consequently, to implement excitation light of a plurality of wavelengths, the present application provides a micro imaging device based on multi-photon. In particular, an input optical fiber connected to the imaging probe and an imaging main unit in the imaging device provided by the present application has a target bandwidth that meets an excitation requirement of a multi-photon imaging, so that a combined laser beam in the target bandwidth is capable of being carried to simultaneously excite multi-photon effects at different wavelengths to observe different internal structures. In addition, for the imaging device, a coupling module of laser beams are set outside the imaging probe, so that the combined laser beam is directly transmitted through the input optical fiber, and thus integration of the imaging probe is improved.

Various non-limiting embodiments of the present application will be introduced in detail below with reference to the accompanying drawings.

### Exemplary Imaging Device

In order to further describe an imaging device that is capable of implementing simultaneous excitation of a plurality of wavelengths provide by the present application, the present application further provided a schematic block diagram (FIG. 2) of the interior of an imaging device, to describe a transmission status of optoelectronic signals (especially laser beams of various wavelengths) in the imaging device.

As shown in FIG. 2, actual function modules in the imaging device may include a processing module, a signal collection module, a scanning control module, a laser device group, an optical detector group, a coupling module, a beam splitting module, and an imaging apparatus.

It should be noted that, considering that the imaging main unit and the imaging probe in the imaging device described above are actually accommodating structures that accommodate a component/module, the imaging main unit and the imaging probe are not shown in the structure shown in FIG. 2.

As shown in FIG. 2, during imaging, the processing module may generate a control signal and apply the control signal to the laser device group. Each laser device in the laser device group may release laser beams with corresponding wavelengths in respond to the control signal and send the laser beams to the coupling module. The coupling module may perform spatial coupling processing on the laser beams to combine the laser beams into a combined laser beam and transmit the combined laser beam to the imaging apparatus. The imaging apparatus may convert the combined laser beam into excitation light that is capable of triggering a multi-photon effect, and apply the excitation light to a specific depth of an imaging object to trigger the multi-photon effect at this depth to collect a corresponding optical signal.

Considering that multi-photon effects triggered by laser beams of different wavelengths generally are presented as optical signals of different wavelengths, optical signals generated after the foregoing excitation light including different components triggers the multi-photon effects often include different wavelengths. Consequently, after collecting the optical signals, the imaging apparatus may transmit the optical signals to the beam splitting module, to decouple components of different wavelengths in the optical signals, so as to determine a plurality of optical signal splitting beams. The optical signal splitting beams may be transmitted to a corresponding optical detector, to sense an intensity of the corresponding signal. The signal collection module may receive a time-series optical signal intensity sensed by each optical detector in time series of the optical detector set, and forward the time-series optical signal intensity to the processing module. The processing module may determine, based on a status of a received optical signal status, a pixel value of a corresponding pixel in an optical image.

Considering that generally only a single point can be excited for multi-photon depth imaging, that is, the imaging apparatus is often required to focus excitation light on a focusing position inside the imaging object during imaging. In order to implement optical imaging in a specific area, in the present application, a focusing position may be changed by an electronic control device in the imaging apparatus. Specifically, a scanning process of multi-photon depth imaging is generally horizontal scanning, that is, the imaging apparatus may focus excitation light on a focusing position of a focal plane inside an imaging object to implement scanning of each point on the focal plane by changing the focusing position.

Therefore, as shown in FIG. 2, the processing module may generate a scanning control signal during imaging, convert the scan control signal into a galvanometer control signal by using the scanning control module, and apply the galvanometer control module to the imaging apparatus, to change a position of the excitation light on the focal plane. A processor may determine, based on the scanning control signal, to align the time-series optical signal intensities with respective position, to render the optical image.

In the present application, a focal plane may further be changed by using the imaging apparatus. In this case, the processing module may further generate a depth control signal, and send the depth control signal to the scanning control module to generate a zoom control signal of the imaging apparatus, so as to control an imaging depth of the imaging apparatus. In some embodiments, three-dimensional reconstruction may be performed based on optical images of different depths to determine a three-dimensional image.

In the present application, the processing module, the signal collection module, the scanning control module, the laser device group, and the coupling module in FIG. 2 may be integrated in the imaging main unit, and the imaging apparatus may be integrated in the imaging probe. Considering separated design of the imaging probe and the imaging main unit, an output optical fiber may be disposed between the imaging main unit and the imaging probe to implement transmission of a combined laser beam.

Further considering an excitation requirement of multi photons, an energy transmission loss of an input optical fiber in a target bandwidth meets an excitation requirement of a multi-photon effect, and a wavelength of each laser beam in the combined laser beam is within the target bandwidth. A specific bandwidth of the input optical fiber may be determined based on specific excitation light of the multi-photon effect.

To achieve the target bandwidth, the input optical fiber meets the excitation requirement of the multi-photon effect in the target bandwidth based on an anti-resonance principle. An anti-resonance principle optical fiber, especially ae hollow-core anti-resonance optical fiber (HC-ARF), is a microstructure optical fiber that implements transmission of an optical signal by using an anti-resonance effect. The basic principle is that, a series of thin layers with a high index and a low refractive are disposed around a core of an optical fiber to form a structure similar to a Fabry-Perot (F-P) resonant cavity, when a wavelength of light meets a specific condition, a constraint effect of the optical fiber on the light weakens, causing the light to quickly escape through the cladding layer to outside, resulting in an increase in transmission loss; on the contrary, when the wavelength of light does not meet the condition, the constraint effect of the optical fiber on the light is enhanced, so that the light propagates along the core, and thus the transmission loss is decreased.

Based on the principle described above, the input optical fiber provided in the present application may include a two-layer structure. Specifically, the input optical fiber may include a silicon core and a cladding layer surrounding the silicon core, and a plurality of air holes arranged at an equal interval are disposed in the cladding layer. The cladding layer in which the air holes are formed may form the structure of the resonant cavity described above, so that there is a strong constraint effect on excitation light within the target bandwidth.

However, in the field of traditional multi-photon imaging (such as multi-photon depth imaging), in order to ensure an excitation power of excitation light, an input optical fiber generally adopts a photonic bandgap hollow-core fiber (PBG-HCF), this type of optical fiber has a good transmission ability (small energy loss and small dispersion) only within a small range of specific wavelengths. Consequently, such optical fiber cannot meet a transmission requirement of the combined laser beam of the present application.

To further illustrate the target bandwidth for transmitting light in the present application, multi-photon imaging is used as an example. In the multi-photon imaging, the excitation light generally ranges from 550 nm to 1800 nm, and thus the input optical fiber provided in the present application may be designed to have good performance within 550 nm-1800 nm.

In particular, considering an imaging requirement of imaging of a brain of a mouse, in depth imaging based on a two-photon fluorescence excitation effect, the target bandwidth of the transmission optical fiber provided in the present application may range from 550 nm to 1100 nm.

To further illustrate performance of the input optical fiber provided in the present application, the present application also provides transmission spectra (FIG. 3A), and a chart shown in FIG. 3A may reflect energy loss of light of different wavelengths passing through different types of optical fibers. In the chart, a broken line with a relative wide range of bandpass indicates the loss of an anti-resonance hollow-core fiber (AR-HCF), a broken line with a relative narrow range of bandpass indicates the loss of photonic bandgap hollow-core fiber (PBG-HCF), the horizontal axis indicates the wavelength, and the vertical axis indicates the loss value.

In addition, FIG. 3A also shows a structural schematic diagram of input optical fibers (namely, AR-HCF in FIG. 3) provided in the present application and a structural schematic diagram of conventional pulse optical fibers (namely, PBG-HCF in FIG. 3).

As can be seen from FIG. 3A, the conventional pulse input optical fiber has a bandpass only at a specific wavelength (for example, the PBG-HCF in FIG. 3 has a bandpass at around 920 nm), while the input optical fiber provided in the present application (namely, the AR-HCF in FIG. 3) has good performance within the range from 550 nm to 1100 nm, so that a target bandwidth range may range from 550 nm to 1100 nm.

As an exemplary illustration only, based on the target bandwidth range, when two-photon fluorescence microscopy is using in the field of imaging of a brain of a mouse, the laser device group includes at least one of a first laser device for releasing a 780 nm laser beam, a second laser device for releasing a 920 nm laser beam, and a third laser device for releasing a 1030 nm beam laser.

780 nm excitation light is used to excite fluorescence information of amyloid plaque in an imaging object. A fluorescence probe of the 780 nm excitation light may be Methoxy-X04, and a wavelength of an optical signal of this probe is generally 460±25 nm. 920 nm excitation light is used for exciting fluorescence information of mitochondria and calcium ion channels thereof in an imaging object. A fluorescence probe of the 920 nm excitation light may be AAV-mito-GCaMP6f+CaMKll-Cre, and a wavelength of an optical signal of this probe is generally 520±35 nm. 1030 nm excitation light is used for exciting fluorescence information of neurons in an imaging object. A fluorescence probe of the 1030 nm excitation optical may be AAV-hSyn-jRGECO1a, and a wavelength of an optical signal of the probe is generally 625±45 nm. For specific content and function mechanism of each fluorescence probe, reference may be made to related technologies, and details are not described herein.

In addition, in practical applications, the output fiber provided in the present application may withstand a high power (there may be higher power in multi-laser devices transmission), and output quality under the high power is not affected. Therefore, when a plurality of laser beams are combined, the output power is relatively high. The output fiber provided in the present application may also meet the transmission requirement in such case. In addition, after optical setting, the dispersion of the output fiber is very small. After being tested by an autocorrelator, it may be learned that the dispersion of the output fiber is basically the same as that of a conventional pulse-type input fiber with single-wavelength, and no additional dispersion of the system is caused.

To further describe transmission of laser beams of different powers, the present application also provides a diagram of energy loss of laser beams of different powers based on the input fiber (FIG. 3B).

In FIG. 3B, “Input fiber” may indicate a pulse width obtained before a laser beam is input into a fiber. Laser beams of different powers are represented by lines of different colors, and specific data may indicate a pulse width obtained after a laser beam is output from the fiber in a corresponding power. As shown in FIG. 3B, the output fiber provided in the present application has a good transmission effect for laser beams of different wavelengths and powers, and can meet the imaging requirements of deep brain imaging.

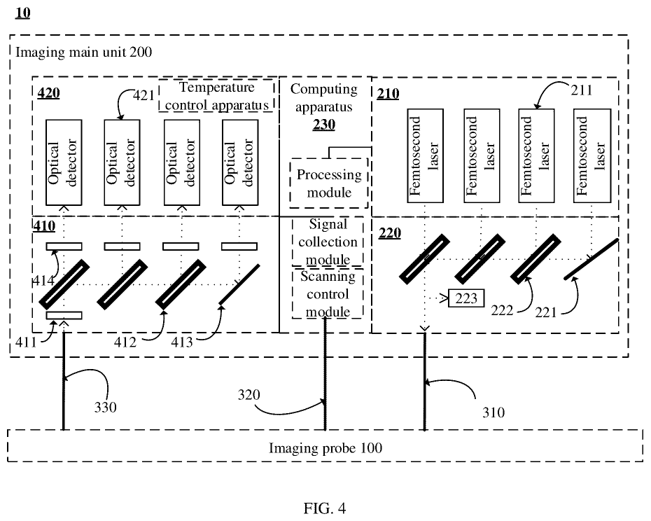

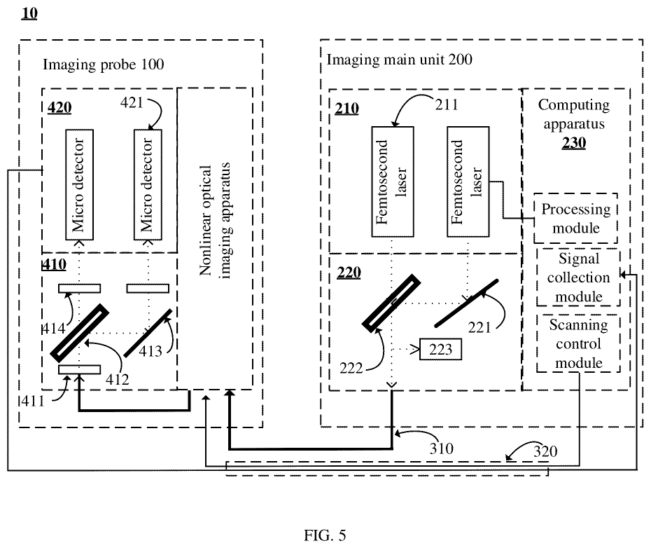

In some embodiments, the beam splitting module 420 and the optical detector group may be disposed in the imaging probe or imaging main unit as required. Considering differences caused by the two setting manners, the present application further provides two system block diagrams of imaging devices (FIG. 4 and FIG. 5). FIG. 4 may show an imaging device in which the beam splitting module and the optical detector group 420 are integrated into the imaging main unit. FIG. 5 may show an imaging device in which the beam splitting module and the optical detector group 420 are integrated into the imaging main unit.

As shown in FIG. 4, the imaging device 10 may specifically include an imaging probe 100, an imaging main unit 200, and a photoelectric composite cable 300. The imaging main unit 200 may at least include a laser device group 210, a coupling module 220, a computing apparatus 230, a beam splitting module 410 and an optical detector group 420. The photoelectric composite cable 300 may at least include an input optical fiber 310 configured to connect the laser device group 210 and the imaging probe 100, a control cable 320 and an output optical fiber 330.

The laser device group 210 may be a set of at least two laser devices 211. The laser devices 211 may release laser beams of different wavelengths that meet a multi-photon imaging requirement. A specific wavelength of a laser device may be determined according to an actually selected multi-photon effect and an imaging purpose, which is not limited herein. In some embodiments, the laser device 211 in the present application may be a femtosecond laser device. For example, the laser device 211 may be a fiber femtosecond laser device, a titanium sapphire femtosecond laser device, or the like.

The coupling module 220 may be an optical element set configured to couple a plurality of laser beams into a combined laser beam. That is, various laser beams released by the laser devices 211 are spatially staggered, and to simultaneously excite multi-photon effects corresponding to different wavelengths, different laser beams are required to be coupled into one beam. In some embodiments, the coupling module 220 may construct a coupling optical path through a conventional beam combining element (such as a dichroic mirror) and a related optical element to make a plurality of laser beams form a combined laser beam after entering the coupling optical path.

The computing apparatus 230 may be a set of devices (such as a processor and a memory) that have a computing capability in the imaging main unit. The computing apparatus 230 may generally include a processing module, a signal collection module and a scanning control module. Considering that the computing apparatus 230 formed by computing devices, the wiring cable related to the computing apparatus is generally realized by an electrical cable.

The imaging probe 100 may have a built-in imaging apparatus that is based on a multi-photon effect and configured to: when being fixedly connected to the imaging object, convert a combined laser beam into excitation light and focus the excitation light at a focusing position in a focal plane inside an imaging object, to trigger a multi-photon effect at the focusing position. Considering that the combined laser beam includes a laser beam of a plurality of wavelengths, the excitation light may simultaneously excite multi-photon effects of different wavelengths.

It should be noted that a specific structure and composition of the imaging apparatus may be modified adaptively depending on an adopted multi-photon effect. Considering that multi-photon-based nonlinear optical effects are generally excited based on a case that a plurality of photons simultaneously focus at a same position, the excitation light may be parallel beams that meet a requirement of multi-photon imaging, and when the parallel beams converges at one point, the multi-photon effects may be triggered to generate an optical signal.

Illustratively, the foregoing imaging apparatus may be constructed based on a second harmonic generation effect or a high-order harmonic generation effect. In this case, the foregoing excitation light may be parallel beams of a specific frequency that meets the requirement of multi-photon imaging, and when the parallel beams converge at one point, harmonic generation may be triggered to generate a new photon of a double frequency or a plurality of frequency. In addition, the foregoing imaging apparatus may alternatively be constructed based on multi-photon effects such as multi-photon excitation fluorescence and coherent anti-Stokes Raman scattering (CARS), and a specific structure may be adjusted according to a corresponding multi-photon effect. The following uses an imaging apparatus based on a two-photon excitation fluorescence effect for description, and other multi-photon imaging effects may be adaptively adjusted.

A control cable 320 may be configured to transmit an electrical signal between the imaging probe 100 and the imaging main unit 200. The control cable 320 may transmit a control signal of a scanning galvanometer to the imaging probe 100 to control a focusing position to implement scanning of each position in a focal plane. Specifically, during depth imaging of multi-photon effects, the excitation light often needs to converge into a focusing position. In this case, to acquire an optical image of a specific region, the imaging probe 100 may control a focusing position of excitation light in a focal plane inside an imaging object during multi-photon imaging, and collect an optical signal of each position in the focal plane by scanning the focal plane. In addition, a control signal of another controllable apparatus (such as an electric zoom module) of the imaging probe 100 may also be transmitted by a control cable.

The output optical fiber 330 is configured to transmit, between the imaging probe 100 and the imaging main unit 200, an optical signal generated by a multi-photon effect. Different from the combined laser beam, considering that the optical signal generated by the multi-photon effect always determines an actual status of a corresponding position by determining whether there is an optical signal (for example, whether there is fluorescence, whether there is a high-frequency signal, or the like), the output optical fiber 330 is generally required to be capable of transmitting an optical signal of a corresponding frequency band, and there is no excessive requirement for performance such as energy loss. Further, considering that the optical signal includes a signal corresponding to laser beams of various wavelengths, it is ok that the output optical fiber 330 may transmit this optical signal. Preferably, in order to reduce an impact of the output optical fiber 330 on free movement of the imaging object, the output optical fiber 330 may be configured to be a collection supple fiber bundle (SFB).

The beam splitting module 410 may be a beam splitting module constructed based on an optical element, and may be configured to split an optical signal based on nonlinear optical effects triggered by a plurality of photons, to determine a plurality of optical signal beams of different wavelengths. Considering that the combined laser beam includes at least two laser beams of different wavelengths, all laser beams of various wavelengths in the multi-photon imaging may trigger a nonlinear optical effect of a corresponding wavelength/frequency band, so that optical signals responding to different wavelengths are included. Therefore, the beam splitting module 410 may split responses of laser beams of various wavelengths in the optical signal to obtain a plurality of optical signal splitting beams.

The optical detector group 420 may be a set of various optical detectors in the imaging main unit 200. An optical detector may be corresponding to an optical signal beam, and is disposed toward a corresponding optical signal splitting beam to detect a signal intensity of the corresponding optical signal splitting beam. In an actual detection process, considering that a scanning process changes quickly, each optical detector in the optical detector group 420 may be configured as a sensor based on an analog signal (such as a photomultiplier tube (PMT)), to output a time-series intensity status of a beam of each optical signal (referred to as a time-series optical signal intensity).

The processing module in the computing apparatus 230 may obtain the foregoing time-series optical signal intensity by the signal collection module, and process the time-series optical signal intensity based on a scanning control signal delivered by the processing module through the scanning control module to determine an optical signal image. In the optical signal image, optical signal splitting beams of different wavelengths may be rendered by different colors, and a pixel value of each optical signal beam in pixels of the image may be determined based on a signal intensity at a corresponding moment. That is, the time-series optical signal intensity on the focal plane is converted into a distribution of an intensity at a focal plane based on the time-series in the scanning control signal, and then is converted into gray/color value based on an intensity to determine an image under the optical signal beam, and images of splitting beams are superimposed to obtain the optical signal image.

In some embodiments, the foregoing computing apparatus 230 or a processing module thereof may further process the optical signal image. For example, an optical signal image may be continuously acquired during actual detection, and then optical signal images based on time series are analyzed. Illustratively in the present application, a transient change curve of calcium ions in body of the mouse may be determined based on the mouse intracranial imaging involving a plurality of laser beams. For another example, the foregoing processing module may further perform three-dimensional spatial reconstruction based on the depth control signal and an optical signal, collected by the imaging probe, corresponding to a corresponding signal, to determine a corresponding three-dimensional model.

As shown in FIG. 4, the laser device group 210 may include four laser devices 211. The laser devices 211 releases different wavelengths of laser beams, and a specific wavelength may be determined according to an imaging requirement of a specific nonlinear optical effect. In addition, the number of lasers 211 may be adjusted according to an actual requirement. For example, in the foregoing description, there may be three laser devices in brain imaging of a mouse, there may be four laser devices 211 in FIG. 4, and there may be two laser devices 211 in FIG. 5.

In some embodiments, in order to implement coupling of laser beams released by different lasers 211, a plurality of coupling elements may be included in the foregoing coupling module 220. The plurality of coupling elements are arranged in directions of corresponding laser devices to form a coupling optical path for outputting a combined laser beam. Laser beams of various wavelengths are spatially overlapped after entering a coupling optical path to form the combined laser beam.

In practical applications, the coupling elements may generally include a reflection element (such as a reflector) configured to change a light propagation path and a beam combining element (such as a dichroic mirror, or a PBS) for performing beam combining. Specifically, for the four laser devices 211, the coupling module 220 may include one coupling reflector 221 and three coupling dichroic mirrors 222. The coupling reflector 221 is disposed towards the laser device 211 at the edge, a coupling dichroic mirror 222 of the intermediate laser device 211 may reflect a corresponding laser and project an upstream laser beam, so that laser beams converge at the downstream. A coupling dichroic mirror corresponding to the laser device 211 in the same propagation direction as the combined laser beam may project a laser beam of the corresponding laser device 211 to make laser beams of the laser devices 211 converge at a same position to form the combined laser light.

It should be noted that the foregoing description is merely an exemplary structure of a coupling optical path, and a specific structure of the coupling optical path may be adjusted according to an actual requirement, and an active coupling element may also be selected according to requirements. For example, beam coupling may be performed by using a polarizing beam splitter (Polarizing Beam Splitter, PBS) and a lens.

In some embodiments, considering that the coupling module 220 of the present application is disposed in the imaging main unit 200, a size and function of the coupling module 220 are not limited by the environment. To ensure coupling accuracy, the coupling optical path may be fed back and adjusted based on a coupling status of the combined laser beam to ensure high accuracy coupling. Consequently, the coupling module 220 may further include a coupling sensor 223, where the coupling sensor 223 is at least disposed at an output end of the combined laser beam in the imaging main unit 200, and is configured to detect position data and/or power data of a corresponding beam.

Illustratively, the coupling sensor 223 may split a portion of light beams from the combined laser beam through a dichroic mirror or another beam splitting element to detect power and position of this portion of the light beams, thereby reflecting a power and position of the combined laser beam.

In some embodiments, in response to a detection result of the coupling sensor 223, the coupling element (such as the coupling reflector 221 and the coupling dichroic mirror 222) may be disposed on a moving mechanism (for example, a deformable mirror frame) to adjust a coupling optical path based on the position data and/or power data, so as to make the excitation light to meet an excitation requirement of a nonlinear optical effect. In practical applications, the process may be configured as a negative feedback process, that is, the moving mechanism may be controlled based on a difference between a target power and an actual power and/or a difference between a target position and an actual position to eliminate the difference.

Similar to the coupling module 220 described above, considering reversibility of light propagation, the foregoing beam splitting module 410 (which may also be referred to as a decoupling module) may also be constructed based on a similar optical element to decouple different wavelength components in an optical signal. That is, the beam splitting module 410 and the coupling module 220 may similarly include a plurality of optical elements to implement decoupling (that is, beam splitting is performed based on wavelength or another optical characteristic). For example, as shown in FIG. 4, the beam splitting module 410 may include a decoupling dichroic mirror 412 and a decoupling reflector 413. An optical path in the beam splitting module 410 is similar to that in the coupling module 220, and details are not described herein again.

In some embodiments, to ensure purity of an optical signal beam, a filter for filtering other light may be included in the beam splitting module 410. Specifically, an ambient filter 411 for filtering ambient light and a beam splitting filter 414 for filtering light other than an optical signal beam may be included.

Similar to the laser group 210 described above, the optical detector group 420 may also be provided with a plurality of optical detectors 421. It should be noted that a wavelength collected by an optical detector 421 and a quantity of optical detectors 421 to be disposed may be adjusted according to an actually dependent non-linear optical effect and an imaging requirement, and are not strictly consistent with the number of laser devices 211.

In some embodiments, considering heat accumulation caused by long-term operation of the optical detector 421, the imaging main unit 200 is further provided with a temperature control apparatus (for example, a semiconductor heat sink), where the temperature control apparatus is at least used for reducing an operating temperature of the optical detector group 420.

In some embodiments, the temperature control apparatus may be disposed in a space in which the optical detector 421 is located to implement heat dissipation on the optical detector 421 by controlling a temperature of the space in which the optical detector 421 is located. In some embodiments, the temperature control apparatus may directly contact each optical detector 421 to directly dissipate heat from the optical detector 421. For example, the temperature control apparatus may be integrated inside the optical detector 421.

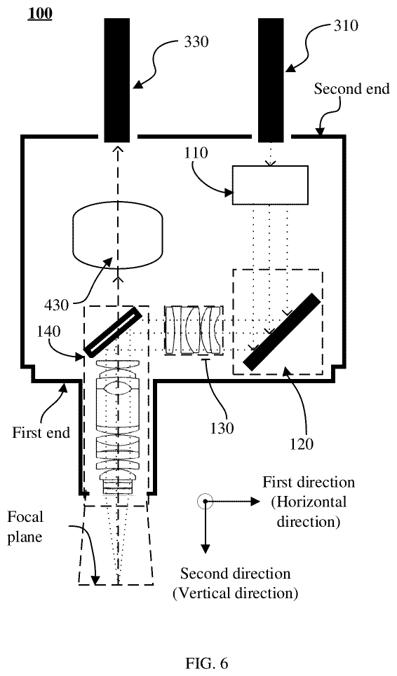

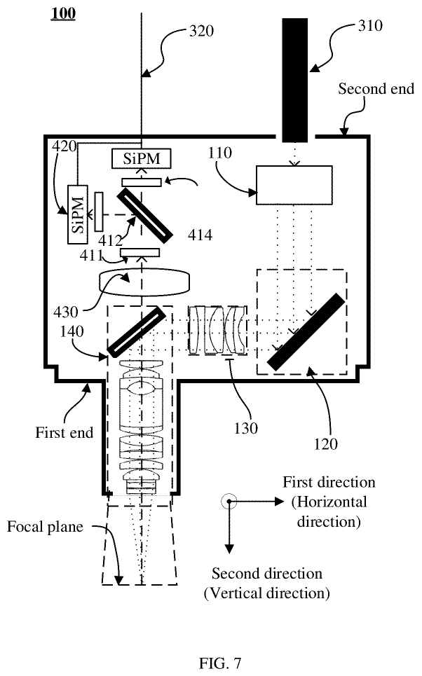

In some embodiments, considering that an optical signal collected in the imaging probe 100 may diverge with propagation in an optical path, and cannot be transmitted by the output optical fiber 330 (or cannot enter the beam splitting module 410 in FIG. 5), a collection apparatus (see the collection lens 430 in subsequent drawings such as in FIG. 6) may be further disposed in the imaging probe 100. The collection apparatus may be constructed based on a lens, so as to perform convergence processing on an optical signal transmitted by the collection apparatus.

Specifically, the lens constituting the collection apparatus may be configured such that a focal point of the optical signal on a surface of the output optical fiber 330 is reduced to 1 mm by using an aspherical lens, thereby allowing use of the output optical fiber 330 with a thinner diameter to minimize obstruction to movement of the imaging object.

As mentioned above, in some embodiments, the beam splitting module 410 and the optical detector group 420 may alternatively be disposed inside the imaging probe 100. For details, referring to FIG. 5.

As shown in FIG. 5, the beam splitting module 410 and the optical detector group 420 may be directly integrated into the imaging probe 100. In this case, the imaging probe 100 does not need to be provided with the output optical fiber 330, and an optical signal is transmitted to the beam splitting module 410 by an internal optical path. In addition, only the time-series optical signal intensity of the optical detector group 420 is required to be transmitted to the signal collection module by a cable (referred to as a transmission cable).

Considering that the multi-photon imaging apparatus in the imaging probe 100 in the present application also needs to be connected to the computing apparatus 230 by a cable, the cable may be reused, so that a transmission cable may be used as a control cable 320 for transmitting a control signal of the multi-photon imaging apparatus, and may also be used for externally transmitting a sensor signal of the optical detector group 420.

In some embodiments, considering that the imaging probe 100 itself is relatively small in size, each optical detector in the optical detector group 420 integrated in the imaging probe 100 may be configured as a micro detector capable of collecting an optical signal intensity, so as to reduce a volume of the imaging probe 100. The micro detector may include but is not limited to a silicon photomultiplier tube (SiPM), a micro photomultiplier tube, a photodiode (PD), an avalanche photodiode (APD), a single photon avalanche diode (SPAD), and the like.

Consequently, the imaging device based on multi-photon depth imaging shown in each of FIG. 1 to FIG. 5 has realized a miniaturized imaging probe by using a separated structure. During imaging, the imaging probe may be fixedly connected to an imaging object, and an optical signal inside the imaging object may be collected by exciting a multi-photon effect inside the imaging object by excitation light to detect an internal status in the imaging object. In particular, an input optical fiber connecting the imaging probe and an imaging main unit in the imaging device provided by the present application has a target bandwidth that meets an excitation requirement of a multi-photon imaging effect, so that a combined laser beam in the target bandwidth is capable of being carried to simultaneously excite multi-photon effects at different wavelengths to observe different internal structures. In addition, for the imaging device, a coupling module of laser beams are set outside the imaging probe, so that the combined laser beam is directly transmitted through the input optical fiber, and thus integration of the imaging probe is improved.

### Exemplary Imaging Probe

In order to further describe optical path propagation in a multi-photon imaging process, the present application further provides schematic diagrams of structures of imaging probes (FIG. 6 and FIG. 7). FIG. 6 and FIG. 7 may present schematic sectional views of imaging probes 100. FIG. 6 may reflect an imaging probe in a case that the beam splitting module 410 and the optical detector group 420 are disposed in an imaging main unit. FIG. 7 may reflect an imaging probe in a case that the beam splitting module 410 and the optical detector group 420 are disposed in the imaging probe.

As shown in FIG. 6, an excitation optical path communicating with a first end to a second end of the imaging probe 100 presents in a body of the imaging probe 100. The first end is used to be fixedly connected to an imaging object, and an input optical fiber is in communication with the imaging probe at the second end to release a combined laser beam at the second end.

Based on the excitation optical path, an imaging apparatus of the imaging probe 100 may include a collimation module 110 disposed at the second end, a scanning module 120 and a lens module 130 that are successively, and an objective lens module 140 disposed at the first end and a collection lens 430.

In the excitation optical path, the collimation module 110 may be configured to convert the combined laser beam into excitation light. The excitation light may simultaneously release a plurality of photons, which may be understood as parallel beams with a same wavelength component. The scanning module 120 may be configured to control an exit angle of the excitation light, the lens module 130 may be configured to transmit the excitation light to the objective lens module 140, and the objective lens module 140 may be configured to focus the excitation light at a focusing position that is in the focal plane inside the imaging object and corresponds to the exit angle.

Specifically, considering a specific principle of multi-photon depth imaging, that is, a plurality of photons may be focused to a same position during imaging to trigger a nonlinear optical effect (such as a fluorescence effect) of the position, in the multi-photon imaging apparatus, the collimation module 110 may convert the combined laser beam into excitation light that includes parallel beams. The parallel beams in the excitation light include all wavelengths in the combined laser beam, and splitting is not performed based on the wavelengths. It should be noted that the parallel beams are only used to indicate that the excitation light simultaneously provides a plurality of photons. In practical applications, specific splitting beams of the excitation light may be configured with different refractive indexes, and thus the beams are not parallel.

As shown in FIG. 4, when the beam splitting module 410 and the optical detector group 420 are disposed in the imaging main unit, the imaging probe 100 is only required to collect a corresponding optical signal and transmit the optical signal to an output optical fiber 330. That is, the imaging probe 100 may further include a collection optical path connected to the first end and the second end, and the output optical fiber 330 is disposed at an opening of the collection optical path at the second end. In this case, an optical signal collected by the objective lens module 140 is directly transmitted to the output optical fiber 330 through the collection optical path. The collection lens 430 may be disposed in the collection optical path to focus the optical signal on a surface of the output optical fiber 330.

Specifically, the output optical fiber 330 may be disposed at a third opening, and the third opening is in communication with the first opening to form a collection optical path to transmit the optical signal to the output optical fiber 330.

In the optical path, the collimation module 110, the scanning module 120, the lens module 130 and the objective lens module 140 each may be a set of optical elements that capable of implementing corresponding functions.

The collimation module 110 (also referred to as a collimator) may be a set of optical elements that collimate the excitation light. That is, a main function of the collimation module 110 is to adjust divergent laser beams to parallel beams. This function is generally implemented by using collimation lenses (or a combination of lenses), and these lenses may transform a combined laser beam approximating to a point light source into a parallel-like collimated beam (namely, the excitation light of the parallel beams).

The scanning module 120 may be a set of a scanning mirror and related optical elements thereof. The scanning module 120 may be configured to perform scanning on a specific region by controlling a propagation direction of light (namely, the excitation light of the parallel beams) through mechanical movement. Specifically, after passing through the scanning module 120, the parallel beams change a propagation direction and an exit angle thereof, so that the excitation light is focused at different positions of an imaging object based on different exit angles by a subsequent module. The scanning mirror in the scanning module 120 is generally a micro-electromechanical system (MEMS)-based scanning mirror or another type of optical component (such as a rotating mirror, a galvanometer, or a prism). Preferably, a scanning galvanometer is subsequently used as a main function device in the scanning module 120.

The lens module 130 may be a set of optical elements configured to transmit excitation light between the scanning module 120 and the objective lens module 140. The lens module 130 is generally a lens combination (for example, a 4f conjugate lens group) to ensure transmission of parallel excitation light.

In some embodiments, considering that the parallel beams with different exit angles in the scanning module 120 have different focusing positions on the focal plane, the lens module 130 may allow the parallel beams with different exit angles to enter the objective lens module 140 at different positions based on a combination of internal lenses, so that the objective lens module 140 is only required to focus light beams at different positions. This may directly ensure that light beams with different exit angles are focused on a same focal plane.

The objective lens module 140 may be a set of optical elements (such as lenses) configured to focus parallel light beams and collect optical signals. The parallel beams entering the objective lens module 140 will converge by each lens in the objective lens module 140, to focus on a specific plane (referred to as a focal plane). In addition, after a multi-photon effect at a focusing position is excited, the focused beams may enter the imaging probe 100 along the objective lens module 140.

Based on the foregoing description and optical path, and considering that different wavelengths may have differences when passing through the optical element, each of the collimation module 110, the lens module 130 and the objective lens module 140 may include at least one group of doublets. The doublet is an optical lens formed by two lenses glued together, and is used for correcting a spherical aberration, a coma aberration, and a chromatic aberration. That is, propagation differences of the excitation light of different wavelengths in the combined laser beam may be eliminated by adjusting a parameter of the doublet.

Specifically, in order to implement multi-color excitation, several groups of achromatic doublets are introduced into the collimation module 110, the lens module 130, and the objective lens module 140. By using various optical material combinations with different dispersion, a system achieves achromatism in an excitation optical band (for example, 760±20 nm-1060±20 nm).

As shown in FIG. 6, when the beam splitting module 410 and the optical detector group 420 are disposed in the imaging main unit, the imaging probe 100 is only required to collect a corresponding optical signal and transmit the optical signal to an output optical fiber 330. That is, the imaging probe 100 may further include a collection optical path connected to the first end and the second end, and the output optical fiber 330 is disposed at an opening (referred to a third opening) of the collection optical path at the second end. In this case, an optical signal collected by the objective lens module 140 is directly transmitted to the output optical fiber 330 through the collection optical path. The collection lens 430 may be disposed in the collection optical path to focus an optical signal on a surface of the output optical fiber 330.

In addition, considering that the objective lens module 140 is reused in the excitation optical path and the collection optical path, a beam splitting element such as a dichroic mirror may be disposed in the objective lens module 140 to implement multiplexing of the optical path without crosstalk.

That is, in FIG. 6, the optical signals collected by the objective lens module 140 may be converged to the output optical fiber 330 through the collecting lens 430, and then transmitted to the imaging main unit. In some alternative embodiments, an electrical signal instead of an optical signal may alternatively be transmitted directly to the imaging main unit. In this case, a beam splitting module 410 and an optical detector group 420 that are used to sense an optical signal are integrated in an imaging probe.

For further description, for a case in which the beam splitting module 410 and the optical detector group 420 are integrated in the imaging probe, refer to FIG. 7. In FIG. 7, the beam splitting module 410 and the optical detector group 420 are integrated in an imaging probe 100.

It should be noted that the beam splitting module 410 may be set according to an actual requirement. In a practical application scenario, a combined laser beam may have excitation light of two wavelengths, and thus two optical detectors may be configured. Correspondingly, the beam splitting module 410 may be directly constructed based on a dichroic mirror, to split an optical signal into two beams. Considering that a scanning module 120 communicates with a computing device through a control cable 320, the control cable 320 may also be mounted on the optical detector group 420 to transmit a sensing signal to the computing device.

In addition, considering that there is no need to transmit an optical signal through an output optical fiber, in FIG. 7, a collection optical path may not communicate at a second end, but only communicates with the inside of the imaging probe 100, to make the optical signal collected by an objective lens module 140 be directly transmitted to a beam splitting module. In FIG. 7, each optical detector in the optical detector group 420 may be configured as the SiPM or another micro detector to reduce a volume of the imaging probe 100.

In some embodiments, considering that only two optical detectors are involved in FIG. 7, the beam splitting module in FIG. 7 may directly include a decoupling dichroic mirror 412 for beam splitting. In order to filter an optical signal with another wavelength, the beam splitting module may further include an ambient filter 411 for filtering ambient light and a beam splitting filter 414 for filtering light other than an optical signal splitting beam. Specifically, considering that the optical signal beam may allow the beam splitting filter 414 to pass through, the ambient filter 411 may not be provided.

In some embodiments, to adjust an imaging effect, the objective lens module 140 may include a plurality of candidate objective lenses. Respective candidate objective lenses in the plurality of candidate objective lenses have different focusing positions for excitation light of a same propagation angle to present different fields of view. Thus, in order to assemble each candidate objective lens, the first end of the imaging probe 100 may be detachably connected with each candidate objective lens. A candidate objective lens currently assembled at the first end may be referred to as a target objective lens.

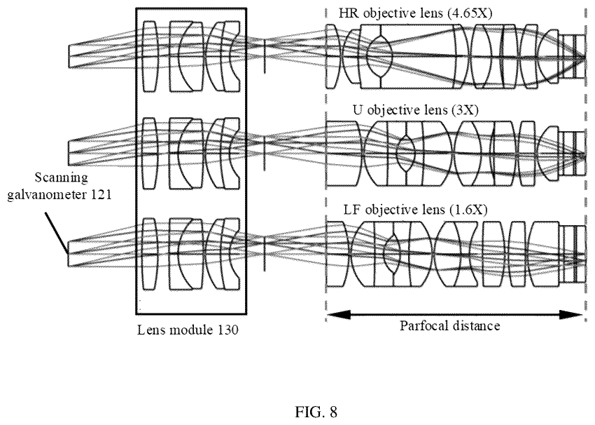

To further describe specific implementation of different ranges of field of view, the present application further provides a schematic diagram of an optical path in an imaging probe based on excitation light of different candidate objective lenses (FIG. 8).

As shown in FIG. 8, a schematic diagram of the optical path may include a scanning galvanometer 121, a lens module 130, and a plurality of candidate objective lenses (respectively referred to as an HR objective lens, a U objective lens, and an LF objective lens). As shown in FIG. 8, the scanning galvanometer 121 may adjust an exit angle of the excitation light, that is, light beams in different colors in FIG. 8 have different exit angles, and enters different candidate objects through a same lens module 130.

Specifically, after light beams of different exit angles in FIG. 8 (specifically presented as lines with different grayscale/color in the figure, or lines with different exit angles exiting from the scanning galvanometer 121 in the figure) are emitted from the scanning galvanometer 121, and may converge to different positions of a candidate objective lens by using the lens module 130, and then the excitation light converged on a point on a focal plane by the candidate objective lens.

Still referring to FIG. 8, it may be learned that, different candidate objective lenses have different convergence positions for excitation light with different exit angles. Specifically, in different candidate objective lenses, the larger a field of view, the smaller a displacement of the angle on the focal plane. If a distance between a convergence position of a light beam of the HR objective lens and a central point is less than a distance, between a convergence position of a light beam of the U objective lens and the central point, that is less than a distance between a convergence position of a light beam of the LF objective lens and the central point, correspondingly, a field of view of the HR objective lens is greater than a field of view of the U objective lens that is greater than a field of view of the LF objective lens.

In some embodiments, parfocal distances (Parfocal Distance) of candidate objective lenses may be the same to ensure that positions of focal planes of the candidate objective lenses for the excitation light are the same. In some embodiments, the objective lens or the first end of the imaging probe is provided with an objective lens length adapter ring and/or a length adjustment apparatus, and the objective lens length adapter ring and/or the length adjustment apparatus is configured to match parfocal distances of different objective lenses.

In some embodiments, to ensure performance of the candidate objective lens and reduce a parameter adjustment process, a common structure and an adjustment structure are included in the candidate objective lens. The common structure is shared among a plurality of candidate objective lenses, the adjustment element is configured to adjust a focusing position of excitation light, and a common element includes at least a doublet. For example, lenses included in the candidate objective lenses in FIG. 8 are the same in type, and only a size is adjusted.

Based on achromatic arrangements such as the doublet, a remaining lateral chromatic aberration is less than 0.5 microns at half of the field of view of each candidate objective lens. For the HR objective lens and U objective lens, the remaining axial chromatic aberration is about 1 micron in the bands 780-920 nm and 920-1030 nm. For the LF objective lens, the remaining axial chromatic aberration is about 1 micron in the band 780-920 nm band and 3 microns in the band 920-1030 nm. In practical applications, a chromatic aberration is extremely small and may be considered as excitation of a nonlinear optical effect at a same position.

In some embodiments, the candidate objective lenses may further be designed for spherical aberration elimination based on practical applications. Specifically, in an actual deep brain imaging process, because a refractive index of the cortex (about 1.38) is higher than a refractive index of the water (about 1.33), the spherical aberration increases with an increase of an imaging depth in a brain tissue. Therefore, the optical path of the HR/U/LF candidate objective lens is optimized to make the spherical aberration introduced in a cortical depth range of 0-800 microns is lower than or slightly higher than a diffraction limit. In addition, at a collection level, the optical path is individually designed such that collection numerical apertures (NA) of the HR, U, and LF targets are expanded to 0.74, 0.67, and 0.4, respectively, to cooperate with the collection lens 430 in the figure to make a focal point of the optical signal on a surface of the output optical fiber 330 be reduced to 1 mm, so that the output optical fiber 330 with a thinner diameter is allowed to use to minimize obstruction to movement of the imaging object.

In practical applications, considering that in the scanning module 120, the excitation light may cause scanning distortion when entering the scanning galvanometer at a large angle, a propagation direction may be adjusted by a reflector (referred to as a scanning reflector) to reduce the angle at which the excitation light enters the scanning galvanometer to make the angle be an acute angle as possible.

To implement that the excitation light enters the scanning galvanometer at an acute angle, there may be two technical directions for design of the interior of the imaging probe. First, at least one reflector (referred to as a scanning reflector) may be disposed between the scanning galvanometer and the collimation module to adjust a propagation angle of the excitation light, to make the excitation light enter the scanning galvanometer at an acute angle. Second, the collimation module and the input optical fiber may be arranged obliquely directly, so that the excitation light itself has a specific inclination angle directly to enter the scanning galvanometer at an acute angle.

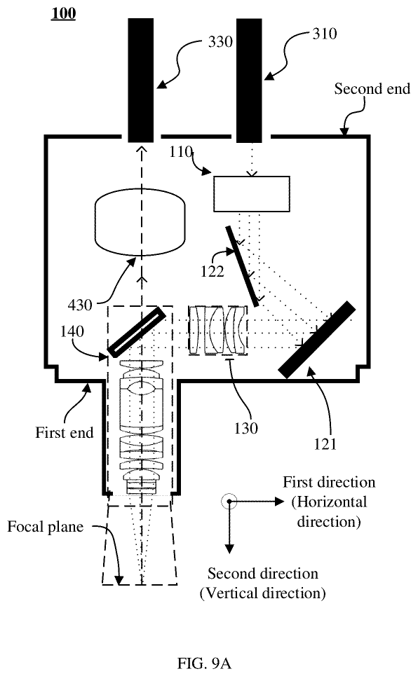

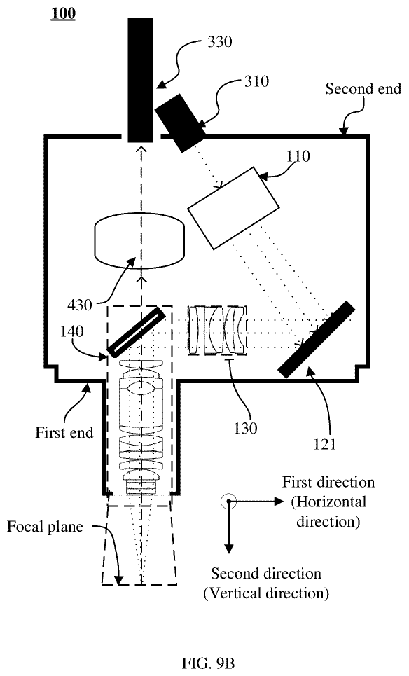

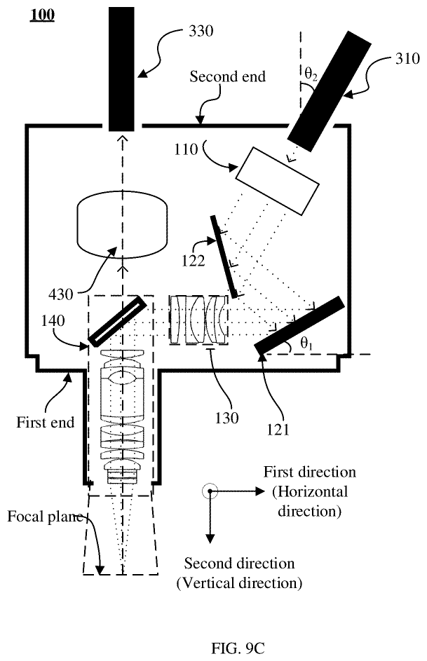

In practical applications, the foregoing technical directions may be combined to form three implementations. To describe the three cases, FIG. 9A to FIG. 9C in the present application illustrate the three implementations.

As shown in FIG. 9A, a scanning module 120 may include a scanning galvanometer 121 and a scanning reflector 122. The scanning reflector 122 is disposed between a collimation module 110 and a scanning galvanometer 121, and is configured to change a propagation direction of the excitation light to make the excitation light reach the scanning galvanometer 121 at an acute angle. A setting angle for the scanning reflector 122 may be adjusted based on positions of the scanning galvanometer 121 and the excitation light, to make the excitation light propagate in a direction shown in the figure.

As shown in FIG. 9B, the scanning module 120 may include only a scanning galvanometer 121. There is a second included angle between an input optical fiber 310 and a second direction (namely, a vertical direction or an objective lens direction) at a second end, to make a combined laser beam enters an imaging probe along the second included angle. Correspondingly, a collimation module is arranged, along the second included angle, downstream of a communication position of an input optical fiber at the second end, and is configured to convert the combined laser beam along the second angle into excitation light along the second included angle. As shown in FIG. 9B, the second included angle may be directly set based on a setting direction of the scanning galvanometer 121 to make the excitation light enter the scanning galvanometer 121 at an acute angle.

With reference to the settings in FIG. 9A and FIG. 9B, in FIG. 9C, the scanning reflector 122 is provided, and there is the second included angle between the input optical fiber 310 and the second direction at the second end, so that the excitation light reaches the scanning galvanometer 121 at an acute angle. In particular, considering an impact of the scanning reflector 122 on propagation, an included angle direction of the second included angle in FIG. 9C is different from that in FIG. 9B.

In particular, scanning distortion may also occur along the included angle of the scanning galvanometer 121 along a first direction. The included angle of the scanning galvanometer 121 along the first direction may be an acute angle (for example, ranging from 15° to 30°). Preferably, an included angle between the scanning galvanometer and the first direction is 20°, and the first direction is an extension direction of a lens module 130.

In some embodiments, in practical applications of multi-photon depth imaging, a focal plane position may also be changed to obtain optical images at different depths for 3D modeling. In practical applications, the change of the focal plane position is usually implemented by using a device such as a displacement platform. However, considering a size of an imaging probe itself, during the process of changing the focal plane position, it is impossible to change the position of the focal plane based on the change of a relative position between the imaging probe and an imaging object realized by using the displacement platform.

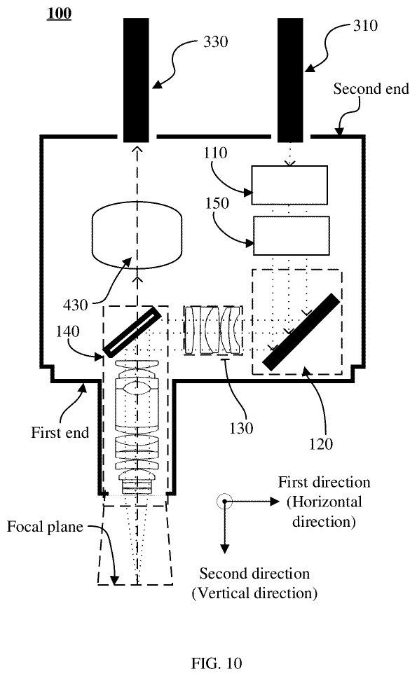

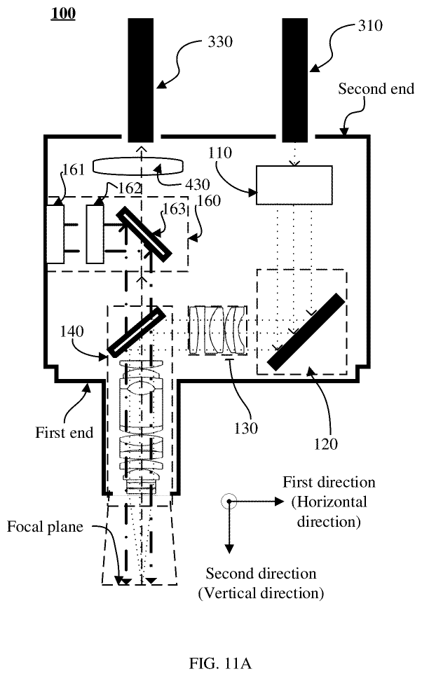

To implement zooming of the imaging probe, the present application creatively changes an internal optical path of the excitation light in the imaging probe, and details are shown in FIG. 10.

As shown in FIG. 10, the imaging probe 100 provided in the present application may be provided with an electric zoom module 150 between the collimation module 110 and the scanning module 120.

The electric zoom module 150 may be an optical device set configured to change a refractive index of the excitation light. For example, the electric zoom module 150 may be constructed by using an electric zoom component. For example, the electric zoom module 150 may be implemented by using an electric zoom component such as a liquid zoom lens or a piezoelectric zoom lens.

That is, considering a principle of multi-photon depth imaging, the excitation light is often configured as parallel beams, and the electric zoom module 150 may change a refractive index of the parallel beams. The refractive index of the parallel beams refers to a refractive ability of an optical system for incident parallel beams, which is used for indicating a degree of focusing or divergence of beams after the beams pass through an optical element.

As shown in FIG. 8, when the excitation light is parallel beams, the objective lens module 140 converge the parallel beams to a corresponding position in the focal plane. When the parallel beams diverge or converge in advance, and as a refractive ability of the objective lens module 140 remains unchanged, so that corresponding excitation light will converge later or in advance, so as to change a depth of the focal plane.

In some embodiments, changes in depth of the focal plane caused by each refractive index may be calibrated in advance. Therefore, in actual control, an electric zoom module may be communicatively connected to the computing apparatus, and a processing module in the computing apparatus may deliver a control signal based on a desired imaging depth and transformation correspondence of the depth described above, to adjust the focal plane depth.

In practical applications of brain imaging of the mouse, a focal plane change ranging from 0 μm to 850 μm may be implemented based on the electric zoom module. Thus, in the scanning process, after completing scanning of one position, the focal plane depth is changed through the electric zoom module, so as to determine fluorescence images of a plurality depths. In this way, the region may be three-dimensionally modeled based on the fluorescence images at various depths. For example, according to the present application, three-dimensional modeling of amyloid plaques, mitochondria, calcium ion channels and neurons in mice within 0 μm-850 μm may be implemented.

In conclusion, the imaging device based on the foregoing design may implement multi-color excitation, imaging depth extension, and switching between targets of different magnifications to achieve an expandable field of view.

In some embodiments, considering that there may be photosensitive proteins (such as ChR2 and NpHR) in an imaging object, optogenetic stimulation may be performed on the photosensitive proteins during the multi-photon depth imaging (especially multi-photon fluorescence excitation), so that specific neurons are activated or inhibited. The optogenetic stimulation generally refers to stimulating a corresponding photosensitive protein by using a light beam with a specific wavelength.

Therefore, considering that an optical image of a cranial nerve (for example, a cranial nerve of a mouse) may be detected according to the present application, an optogenetic light beam is applied simultaneously during imaging to implement optogenetic stimulation.

For example, during the foregoing two-photon brain imaging, a 635 nm light emitting diode (LED) light source may be used to stimulate the inhibitory optogenetic protein ChR2. In this case, the two-photon fluorescence excitation may use a 920 nm light beam to perform imaging of a GCaMP green fluorescent indicator. In addition, to avoid an impact of the LED light source, during collecting a fluorescence signal, a filter (such as the ambient filter 411) may be used at the beam splitting module for filtering out the 635 nm LED light.

For another example, in the two-photon brain imaging, a 488 nm LED light source may be used to stimulate the excitatory optogenetic protein NpHR. In this case, two-photon uses a 1030 nm light beam to perform imaging of a the jRGECO red fluorescent indicator. In addition, to avoid an impact of the LED light source, during collecting a fluorescence signal, a filter (such as the ambient filter 411) may be used at the beam splitting module for filtering out the 488 nm LED light.

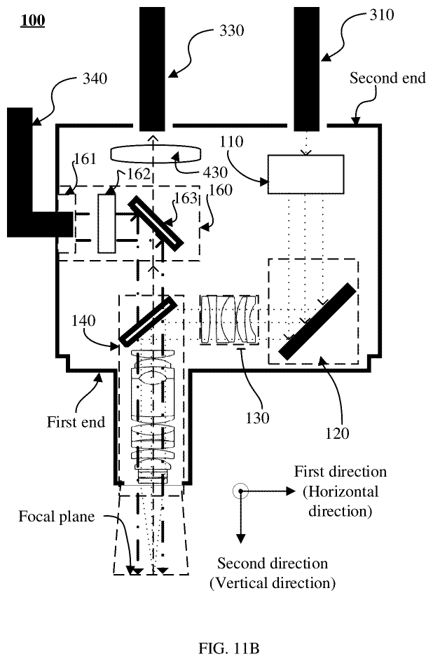

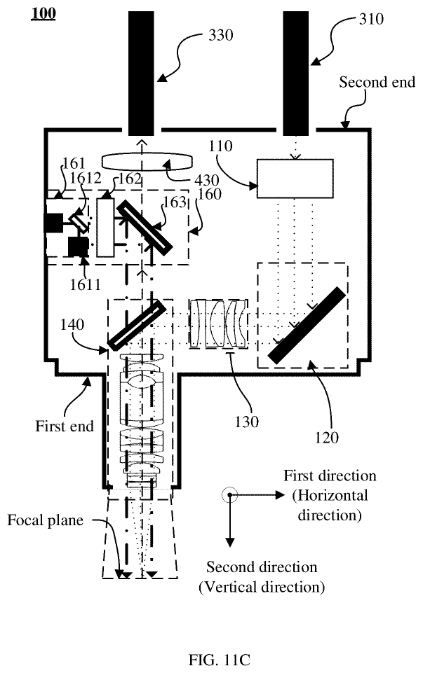

To implement the optogenetic stimulation, the imaging probe 100 may further be provided with an optogenetic module. To describe a specific structure of the optogenetic module, the present application further provides a plurality of schematic structural diagrams (as shown in FIG. 11A-FIG. 11C).

Referring to FIG. 11A, the imaging probe 100 may further include an optogenetic module 160. The optogenetic module 160 is configured to release optogenetic light beams and converge the optogenetic light beams into an excitation optical path at the objective lens module 140, to form an optogenetic stimulation region at a focusing depth of a focal plane, where the optogenetic stimulation region covers the focal plane. That is, the optogenetic light beams may be irradiated in a focal plane region simultaneously with the excitation light. Considering an implementation principle of the optogenetic stimulation, to ensure that all positions in the focal plane are stimulated by the optogenetic stimulation, the optogenetic stimulation region may include the focal plane region formed by the excitation light.

In some embodiments, to implement generation of the optogenetic light beam, the optogenetic module 160 may include an optogenetic light source 161, an optogenetic focusing element 162 (for example a lens or lenses group having a beam focusing function), and an optogenetic input element 163. The optogenetic light source 161 is configured to release optogenetic beams, the optogenetic focusing element 162 is configured to converge the optogenetic beams in optogenetic optical fibers, and the optogenetic input element 163 is configured to input the optogenetic beams to an objective lens module 140 based on the objective lens module 140.

Considering that the optogenetic light beams cover a focusing region of the excitation light at a focal plane, the optogenetic light source may generally be configured as a surface light source. Specifically, in FIG. 11A, the optogenetic light source 161 may be an optogenetic laser device that is capable of releasing an optogenetic light beam. For example, the optogenetic light source 161 may be an LED light source with a specific wavelength. Further considering divergence of the surface light source, the optogenetic focusing element 162 may converge the optogenetic optical fibers to meet an optogenetic stimulation requirement.

The optogenetic input element 163 may be constructed based on a conventional light beam combining element. As shown in FIG. 11A, the optogenetic input element 163 may be configured as a dichroic mirror. Considering that the optogenetic input element 163 overlaps with an optical signal optical path, the dichroic mirrors may be configured based on a wavelength of an optogenetic beam to make the dichroic mirrors reflect the optogenetic beam into the objective lens module 140. Specifically, the optogenetic input element 163 is generally disposed between the collection lens 430 and the objective lens module 140.

In some embodiments, considering that optogenetic light beams of different wavelengths may be used in the optogenetic stimulation (for example, two different light beams are used in the foregoing brain imaging scenario), the optogenetic light source 161 is capable of providing at least two optogenetic light beams to match application in the scenario.

In this case, the optogenetic light source 161 shown in FIG. 11A may be configured as a replaceable optogenetic laser. The replaceable optogenetic laser is detachably connected to an optogenetic beam imaging probe 100, and is configured to provide a corresponding optogenetic beam. That is, during replacement of the wavelength of the optogenetic beam, the optogenetic light source 161 may be replaced with a replaceable optogenetic laser device with a corresponding wavelength through detachable connection.

In some embodiments, to implement release of a plurality of optogenetic light beams, the optogenetic light source 161 may further be configured as an optogenetic optical fiber and an optogenetic laser group. FIG. 11B reflects a structure in which the optogenetic light source is configured as the optogenetic optical fiber, and FIG. 11C reflects a structure in which the optogenetic light source is configured as an optogenetic laser group.

As shown in FIG. 11B, an optogenetic optical fiber 340 may further be disposed on the imaging probe 100. A portion of the optogenetic optical fiber 340 in the imaging probe 100 may be abstracted as the optogenetic light source 161. The optogenetic optical fiber 340 may be connected to a plurality of optogenetic laser devices in an imaging main unit. During optogenetic stimulation, an optogenetic beam of a corresponding wavelength may be released by controlling operation of a corresponding optogenetic laser device.

As shown in FIG. 11C, an optogenetic laser device group (namely, a plurality of composite optogenetic laser devices) may alternatively be directly disposed in the imaging probe 100. Specifically, the optogenetic light source 161 may include a plurality of optogenetic laser devices 1611 and one optogenetic beam combining element 1612. The optogenetic laser device 1611 may release optogenetic beams of different wavelengths, and the optogenetic beam combining element 1612 may combine the optogenetic beams released by various optogenetic laser devices 1611 to a specified position. In practical applications, for a specific beam combining structure of the optogenetic beam combining element 1612, reference may be made to related descriptions of the coupling module 220, and both are the same in principle. For example, the optogenetic beam combining element 1612 may be configured as a dichroic mirror.

All of the foregoing optional technical solutions may be randomly combined to form an optional embodiment of the present application, and details are not described herein.

Those of ordinary skill in the art may be aware that units and algorithm steps in examples described with reference to the embodiments disclosed in this specification can be implemented by an electronic hardware or a combination of a computer software and the electronic hardware. Whether the functions are executed by hardware or software depends on particular applications and design constraints of the technical solutions. A person skilled in the art may use different methods to implement the described functions for each specific application, but it should not be considered that the implementation goes beyond the scope of the present application.

A person skilled in the art may clearly understand that, for convenience and brevity of description, reference may be made to corresponding processes in the foregoing method embodiment for specific working processes of the foregoing systems, apparatuses, and units. Details are not described herein again.

In several embodiments provided in the present application, it should be understood that the disclosed system, apparatus, and method may be implemented in other manners. For example, the described apparatus embodiments are merely examples. For example, the unit division is merely logical function division and may be other division in actual implementation. For example, a plurality of units or components may be combined or integrated into another system, or some features may be ignored or not executed. In addition, the displayed or discussed mutual couplings or direct couplings or communication connections may be implemented as indirect couplings or communication connections through some interfaces, apparatuses or units, and may be implemented in electrical, mechanical, or other forms.

The units described as separate parts may be or may not be physically separate, and parts displayed as units may be or may not be physical units, and may be at one location, or may be distributed on a plurality of network elements. Some or all of the units may be selected according to actual requirements to achieve the objective of the solutions of embodiments.

It should be noted that, in the description of the present application, the terms “first”, “second”, “third”, and the like are merely intended for a purpose of description, and shall not be understood as an indication or implication of relative importance. In addition, in the descriptions of the present application, unless otherwise stated, “a plurality of” means at least two.

It should be noted that, “miniaturization” in the present application means that a multi-photon microscopy system has little impact on activity of a to-be-detected living body during detection of the to-be-detected living body. For example, when the multi-photon microscopy system is fastened to the to-be-detected living body, the to-be-detected living body may still move freely.

The above descriptions are merely preferred embodiments of the present application and are not intended to limit the application. Any modification, equivalent replacement, and the like made within the spirit and principle of the present application shall fall within the protection scope of the present application.

What is claimed is:

1. An imaging device based on multi-photon depth imaging, wherein the imaging device comprises at least two laser devices, a coupling module, an input optical fiber, an imaging probe, a beam splitting module, at least two optical detectors and an imaging main unit;
the imaging main unit at least accommodates the at least two laser devices and the coupling module, the laser devices are configured to provide laser beams meeting an imaging requirement, the coupling module is configured to couple laser beams released by the at least two laser devices into a combined laser beam, wherein laser beams released by laser devices in the at least two laser devices have different wavelengths;
an end of the input optical fiber is connected to the coupling module and the other end is connected to the imaging probe for transmitting the combined laser beam to the imaging probe, wherein an energy transmission loss of the input optical fiber in a target bandwidth meets an excitation requirement of a multi-photon effect, and a wavelength of each laser beam in the combined laser beam is within the target bandwidth;
the imaging probe is internally provided with an imaging apparatus that is based on multi-photon depth imaging and configured to: convert the combined laser beam into excitation light; focus the excitation light at an internal focusing position of an imaging object to trigger a multi-photon effect at the internal focusing position; collect optical signals generated by the multi-photon effect; and change the internal focusing position to determine optical signals of various positions in a focal plane, wherein the optical signals of various positions in the focal plane are used for generating an optical image of the imaging object at the focal plane; and
the beam splitting module is configured to split the optical signals into at least two optical signal splitting beams, and the optical detector is configured to receive a corresponding optical signal splitting beam and detect a signal intensity of the corresponding optical signal beam, wherein the optical signal splitting beams reflect signal components of the optical signals at various wavelengths generated by the multi-photon effect, the optical signal splitting beams are in one-to-one correspondence with the optical detectors, and a signal intensity of the optical signal reflects a pixel value of a corresponding pixel in the optical image.

2. The imaging device according to claim 1, wherein the input optical fiber comprises a silicon core and a cladding layer surrounding the silicon core, a plurality of air holes arranged at an equal interval are disposed in the cladding layer, and the input optical fiber meets the excitation requirement of the multi-photon effect in the target bandwidth based on an anti-resonance principle.

3. The imaging device according to claim 2, wherein the imaging device is configured to image based on a two-photon fluorescence effect, and the target bandwidth of the input optical fiber ranges from 550 nm to 1800 nm.

4. The imaging device according to claim 3, wherein the imaging device is configured as a two-photon microscope, and the at least two laser devices comprise at least one of a laser device for releasing a 780 nm laser beam, a laser device for releasing a 920 nm laser beam, or a laser device for releasing a 1030 nm laser beam,
wherein the 780 nm laser beam is used for exciting fluorescence information of an amyloid plaque in a living body to be detected, the 920 nm laser beam is used for exciting fluorescence information of mitochondria and calcium ion channels of the mitochondria in the living body to be detected, and the 1030 nm laser beam is used for exciting fluorescence information of neurons in the living body to be detected.

5. The imaging device according to claim 1, wherein there is an excitation optical path communicating with a first end and a second end of the imaging probe in the imaging probe, the first end is configured to be fixedly connected to the imaging object, and the input optical fiber is in communication with the imaging probe at the second end and is used for releasing the excitation light at the second end;
the imaging apparatus comprises a collimation module, a scanning module, a lens module and an objective lens module disposed on the first end that are successively disposed at the second end along a propagation direction of the excitation light; in the excitation optical path, the collimation module is configured to convert the combined laser beam into the excitation light, the scanning module is configured to control an exit angle of the excitation light, the lens module is configured to transmit the excitation light to the objective lens module, and the objective lens module is configured to focus the excitation light at a focusing position that is in the focal plane inside the imaging object and corresponds to the exit angle; and
the collimation module, the lens module and the objective lens module each comprise at least one group of doublets for eliminating propagation differences of laser beams of different wavelengths in the excitation light.

6. The imaging device according to claim 5, wherein the objective lens module is detachably connected to the first end; and
the objective lens module comprises a plurality of candidate objective lenses, and respective candidate objective lenses in the plurality of candidate objective lenses have different focusing positions for excitation light of a same propagation angle to present different fields of view.

7. The imaging device according to claim 6, wherein a parfocal distance adjustment apparatus is disposed on the candidate objective lenses or the first end of the imaging probe, wherein the parfocal distance adjustment apparatus comprises an objective lens length adapter ring and/or a length adjustment apparatus, and focal planes of the candidate objective lenses are the same during imaging.

8. The imaging device according to claim 5, wherein the imaging probe further comprises a collection module disposed on an end, away from the imaging object, of the objective lens module, and the collection module is configured to focus optical signals collected by the objective lens module.

9. The imaging device according to claim 5, wherein the scanning module comprises a scanning galvanometer, the scanning galvanometer is configured to control the exit angle of the excitation light, and an included angle between the scanning galvanometer and the excitation light at the scanning galvanometer is an acute angle; and
the scanning module further comprises a scanning reflector, wherein the scanning reflector is disposed between the collimation module and the scanning galvanometer, and is configured to change a propagation direction of the excitation light; and/or
there is a second included angle between a second direction and the input optical fiber at the second opening, so that the combined laser beam enters the imaging probe along the second included angle, and the collimation module is arranged, along the second included angle, downstream of a communicating place of the input optical fiber at the second end, to convert the combined laser beam into excitation light along the second included angle, wherein the second direction is an extension direction of the objective lens module.

10. The imaging device according to claim 9, wherein an included angle between the scanning galvanometer and a first direction is 20°, and the first direction is an extension direction of the lens module.

11. The imaging device according to claim 5, wherein the imaging apparatus further comprises an electric zoom module disposed between the collimation module and the scanning module, and the electric zoom module is configured to change a refractive index of the excitation light and adjust a focal plane depth of the excitation light to determine optical signals at different focal plane depths, wherein the optical signals at different focal plane depths are used for determining a three-dimensional model.

12. The imaging device according to claim 5, wherein the imaging probe further comprises an optogenetic module, the optogenetic module is configured to: release optogenetic light beams, converge the optogenetic light beams into the excitation optical path at the objective lens module, and form an optogenetic stimulation region at a focusing depth of the focal plane, wherein the optogenetic stimulation region covers the focal plane; and
the optogenetic module comprises an optogenetic light source, an optogenetic focusing element, and an optogenetic input element that are successively disposed, wherein the optogenetic light source is configured to release the optogenetic light beams, the optogenetic focusing element is configured to converge the optogenetic light beams, and the optogenetic input element is disposed based on the objective lens module and configured to input the optogenetic light beams into the objective lens module.

13. The imaging device to claim 12, wherein the optogenetic light source is capable of providing at least two types of optogenetic light beams, and the optogenetic light source is configured as one of an optogenetic optical fiber, a replaceable optogenetic laser device, and an optogenetic laser device group; and
when the optogenetic light source is configured as the optogenetic optical fiber, the optogenetic optical fiber is connected to at least two optogenetic laser devices in the imaging main unit for transmitting at least two types of optogenetic light beams;
when the optogenetic light source is configured as the replaceable optogenetic laser device, the replaceable optogenetic laser device is detachably connected to the imaging probe to provide a corresponding optogenetic light beam;
when the optogenetic light source is configured as the optogenetic laser device group, the optogenetic laser device group comprises at least two optogenetic laser devices and at least one beam combining element.

14. The imaging device according to claim 12, wherein the beam splitting module comprises a filter for filtering the optogenetic light beam.

15. The imaging device according to claim 1, wherein the beam splitting module and the at least two optical detectors are arranged inside the imaging main unit, and the imaging device further comprises an output optical fiber;
the output optical fiber separately communicates with the second end of the imaging probe and the beam splitting module, and is configured to transmit an optical signal collected by the imaging probe to the beam splitting module; and
the imaging main unit is further provided with a temperature control apparatus, wherein the temperature control apparatus is at least used for reducing an operating temperature of the at least two optical detectors.

16. The imaging device according to claim 1, wherein the beam splitting module and the at least two optical detectors are arranged inside the imaging probe;
the optical detector is configured as a micro detector, and the imaging probe further comprises transmission cables connected to respective optical detectors, and the transmission cables are configured to transmit signal intensities of respective optical signal splitting beams at a current moment.

17. The imaging device according to claim 16, wherein the transmission cables are integrally disposed in a control cable, and the control cable is connected to the imaging probe and the imaging main unit and configured to transmit a control signal to a controllable device in the imaging probe.

18. The imaging device according to claim 1, wherein the coupling module comprises a plurality of coupling elements and a coupling sensor;
the plurality of coupling elements are arranged in directions of corresponding laser devices to form a coupling optical path for outputting the combined laser beam; and the coupling sensor is at least disposed at an output end of the combined laser beam in the imaging main unit, and is configured to detect position data and/or power data of a corresponding beam; and
the coupling element is disposed on a moving mechanism and configured to adjust the coupling optical path based on the position data and/or power data to make the excitation light meet an excitation requirement of a multi-photon effect.

19. An imaging probe, wherein the imaging probe is applied to an imaging device based on multi-photon depth imaging, the imaging probe comprises a built-in imaging apparatus that is based on multi-photon depth imaging and an input optical fiber connected to the imaging probe;
the input optical fiber is configured to transmit, to the imaging probe, a combined laser beam provided by the imaging main unit, wherein an energy transmission loss of the input optical fiber in a target bandwidth meets an excitation requirement of a multi-photon effect, and the combined laser beam comprises a plurality of laser beams whose wavelength is within the target bandwidth;
the imaging apparatus is configured to: convert the combined laser beam into excitation light; focus the excitation light at an internal focusing position of an imaging object to trigger a multi-photon effect at the internal focusing position; collect optical signals generated by the multi-photon effect; and change the internal focusing position to determine optical signals of various positions in a focal plane, wherein the optical signals of various positions in the focal plane are used for generating an optical image of the imaging object at the focal plane; and
the optical signals form at least two optical signal splitting beams after beam splitting processing, and are received by corresponding optical detectors to determine signal intensities of respective optical signal beams, wherein the optical signal splitting beams reflect signal components of the optical signals at various wavelengths generated by the multi-photon effect, and a signal intensity of the optical signal reflects a pixel value of a corresponding pixel in the optical image.

20. The imaging probe according to claim 19, wherein the input optical fiber comprises a silicon core and a cladding layer surrounding the silicon core, a plurality of air holes arranged at an equal interval are disposed in the cladding layer, and the input optical fiber meets the excitation requirement of the multi-photon effect in the target bandwidth based on an anti-resonance principle.
